(19) United States
(12) Patent Application Publication
(10) Pub. No.: US 2026/0207839 A1
(43) Pub. Date: Jul. 23, 2026

(54) INFUSION PUMP WITH MAGNETIC KEYED LOCK

(71) Applicant: Fresenius Vial SAS, Brézins (FR)

(72) Inventors: Mathieu Paoli, La Murette (FR); Guillaume Loussert, Virville (FR); Florent Brault, Mery (FR); Romain Mollard, Saint-Georges d'Espéranche (FR); Gregory Daubelcourt, Brézins (FR)

(21) Appl. No.: 19/142,511

(22) PCT Filed: Mar. 8, 2024
(86) PCT No.: PCT/EP2024/056175

§ 371 Date: Jun. 23, 2025

(30) Foreign Application Priority Data
Mar. 10, 2023 (EP) 23315043.2

(51) Int. Cl. A61M 5/142; A61M 5/14

(52) U.S. Cl. CPC A61M 5/142; A61M 5/1415; A61M 2005/1405; A61M 2202/0007; A61M 2202/0241; A61M 2205/0272; A61M 2205/276; A61M 2209/082

(57) ABSTRACT

An infusion pump for analgesic treatment, in particular a patient-controlled analgesia pump, is provided. The infusion pump comprises at least one lock preventing unauthorized handling of the infusion pump. The lock is a magnetic keyed lock. Further, a magnetic key for an infusion pump and a system comprising an infusion pump and a magnetic key are provided.

The invention relates to an infusion pump, in particular a patient-controlled analgesia pump, comprising at least one lock preventing unauthorized handling of the infusion pump, a key for an infusion pump as well as a system comprising an infusion pump and a corresponding key.

Infusion pumps are used to administer medication, anesthetics and analgesia in a controlled and automated way. The medication, anesthetic or analgesic to be administered is stored in a compartment of the pump. The medication, anesthetic or analgesic is provided, for example, in a syringe which is stored in the pump compartment. In order to prevent unauthorized handling of such infusion pumps, security mechanisms are provided on the pump. Usually, the infusion pump is secured to a pole in order to prevent theft of the pump. Further, the pump compartment is protected from unauthorized access in order to prevent theft of the medication, anesthetic or analgesic provided therein. Preventing unauthorized access is particularly important for analgesic treatment where patients may want to increase the administered dose beyond their allowed and healthy dose and bolus.

In the prior art, mechanical key locks are used to prevent unauthorized handling of the pump, e.g. a mechanical lock is provided in order to secure the pump to a pole (pole-lock mechanism) and/or in order to lock the pump compartment. In the locked state of the mechanical key lock, a system of cam or wheel, levers and springs prevents removal of the infusion pump from the pole (pin tumbler lock). Similarly, a door of the pump compartment cannot be opened in a locked state of a key lock. Inserting and turning a suitable key in the key lock opens the lock and releases the corresponding security mechanism: the infusion pump can be removed from the pole or the medication, anesthetic or analgesic be retrieved from the pump compartment, respectively.

It is a disadvantage of present systems that the mechanical lock can be pried open through abuse of tools that are available in a hospital environment, such as knifes. Unauthorized access cannot be prevented in these cases. Replacement of these locks is complex. Moreover, when multiple pumps are arranged on top of each other on a pole or in a rack of an infusion pump station or stacked on top of each other otherwise, mechanical locks can be difficult to access such that inserting and turning the key is cumbersome. Further, contact with liquids present in a hospital environment can lead to corrosion that makes the lock unusable. Another issue is hygiene: it is difficult to keep the lock free of germs.

It is an objective of the present invention to overcome the above-mentioned deficits of the present state of the art and provide an infusion pump, a key and corresponding system that increase patient safety, are easy to handle and more hygienic.

This objective is achieved by means of an infusion pump, in particular a patient-controlled analgesia pump, for analgesic treatment, comprising at least one lock preventing unauthorized handling of the infusion pump, wherein the magnetic keyed lock has a locked state and an unlocked state and wherein the magnetic keyed lock is configured to switch from the locked state to the unlocked state through placement of a magnetic key on the magnetic keyed lock or in the vicinity of the magnetic keyed lock.

The infusion pump is, for example, a syringe infusion pump comprising a pump compartment in which a syringe can be provided. In particular, the infusion pump is used for analgesic treatment. For example, it comprises a pump compartment in which a syringe comprising an analgesic can be arranged and its content administered by the pump. When in use, the infusion pump is usually clamped to a pole, e.g. at the bedside of a patient. In order to prevent unauthorized handling of the pump, either or both, the fastening mechanism of the pump to the pole and the pump compartment have to be secured. A lock secures the pump from unauthorized removal from a pole on which it is fastened, for example. Additionally or alternatively, a lock is provided which secures the pump compartment against unauthorized access. Hence, the pump can comprise multiple, e.g. two, locks. Unauthorized handling comprises the handling by unauthorized persons, such as patients or visitors. The at least one lock prevents unauthorized handling in particular in a hospital setting. A magnetic keyed lock is a lock that in contrast to a mere mechanical lock relies on magnetic forces to implement the locking and unlocking mechanism. Through placement of a suitable or corresponding magnetic key on the magnetic keyed lock or in the vicinity of the magnetic keyed lock, a defined magnetic field is provided at the magnetic keyed lock. The resulting magnetic forces lead to an unlocking of the magnetic keyed lock.

In one embodiment, the magnetic keyed lock comprises an engagement member connected with a housing of the infusion pump and wherein the magnetic keyed lock comprises a locking member connected with a movable part of the infusion pump, wherein the engagement member engages with the locking member in a first, locked state of the magnetic keyed lock and releases the locking member in a second, unlocked state of the magnetic keyed lock.

In one embodiment, the engagement member comprises at least one ferromagnetic element, wherein the ferromagnetic element comprises a hard ferromagnetic material and/or a soft ferromagnetic material. Through the at least one ferromagnetic element, the engagement member is able to react to the application of a defined magnetic field, e.g. through the placement of a magnetic key. Multiple ferromagnetic elements can be provided in one example. The ferromagnetic element is, for example, a permanent magnet. Alternatively, the ferromagnetic element is, for example, an element made of a soft ferromagnetic material such as e.g. iron.

In one embodiment, the engagement member is movably connected to the housing in such a way that the engagement member switches between a first position corresponding to the locked state of the magnetic keyed lock and a second position corresponding to the unlocked state of the magnetic keyed lock through placement of a magnetic key on the magnetic keyed lock or in the vicinity of the magnetic keyed lock. Hence, unlocking of the magnetic keyed lock is achieved through the application of a defined magnetic field provided by the magnetic key. The defined magnetic field is the magnetic field under the application of which the engagement member switches from the first position into the second position, hence achieving an unlocking of the lock. In particular, the defined magnetic field has to be strong enough in order to overcome the forces that hold the engagement member in the first position, e.g. gravitational forces, frictional forces and/or spring forces. For example, the engagement member is kept in the first position by a spring, which is compressed when the engagement member is moved into the second position.

In one embodiment, the engagement member is rotatably connected to the housing in such a way that the engagement member is rotatable between the first position and the second position. The rotation is achieved through placement of a magnetic key on the magnetic keyed lock or in the vicinity of the magnetic keyed lock. In particular, the engagement member rests in the first position, when the no magnetic key is placed on the magnetic keyed lock or placed in the vicinity of the magnetic keyed lock, in particular when a defined magnetic field is absent,, and switches into the second position, when the a magnetic key is placed on the magnetic keyed lock or placed in the vicinity of the magnetic keyed lock, in particular when a defined magnetic field is applied. Through placement of the magnetic key on or in the vicinity of the magnetic keyed lock, in particular through the application of a defined magnetic field, the magnetic keyed lock is unlocked, while it remains locked as long as no magnetic field is applied.

In one embodiment, the engagement member comprises a first protrusion and the locking member comprises at least one recess or at least one opening. The first protrusion engages with the at least one recess or with the at least one opening in the locked state of the magnetic keyed lock. The first protrusion is removed from the at least one recess or from the at least one opening in the unlocked state of the magnetic keyed lock. The removal of the first protrusion from the at least one recess or from the at least one opening is e.g. achieved through a rotation of the engagement member, wherein the rotation is initiated by a placement of a magnetic key on or in the vicinity of the magnetic keyed lock, in particular by the application of a defined magnetic field.

In one embodiment, the at least one lock is a lock for a pump compartment of the infusion pump, in particular a syringe infusion pump, wherein the pump compartment is part of the housing of the infusion pump and the movable part is a door of the pump compartment. The pump compartment is a compartment provided in the housing in which a syringe, e.g. for medication, anesthetics or analgesics, can be arranged. The pump compartment is secured with a door in order to prevent access to the syringe by unauthorized persons, e.g. patients. The pump compartment is e.g. provided at the front of the infusion pump. In particular, the door as movable part of the infusion pump comprises a locking member. The locking member is provided for example with one recess or opening with which a protrusion of the engagement member engages. In this example, the engagement member comprises a ferromagnetic element, e.g. a permanent magnet arranged on the top of the engagement member. In the locked position, a protrusion of the engagement member engages with the recess or opening of the locking member. If a magnetic key is placed on the magnetic keyed lock or in its vicinity, in particular if a defined magnetic field is applied, the engagement member is lifted such that the protrusion is removed from the recess or from the opening, respectively. The door of the pump compartment can be opened. If the magnetic key and hence the defined magnetic field is removed, the engagement member drops under the influence of the gravitational force or a spring force. For example, the engagement member is kept in the first position by a spring, which is compressed when the engagement member is moved into the second position. If the door is closed, the protrusion re-engages with the recess or with the opening, respectively. An opening of the door is not possible now. By securing the pump compartment with a magnetic keyed lock, the pump compartment can no longer be pried open by simple tools such as knifes since the lock is no more visible and physically accessible to the user. The safety of pump use is thus increased as unauthorized access is made more difficult or even impossible with the tools available in a standard hospital environment. Moreover, opening of the pump compartment by authorized personal is made more comfortable as the lock merely has to be approached by a magnetic key. No turning of a key in a confined space is necessary. Swift and easy cleaning of the housing comprising the pump compartment is made possible.

In one embodiment, the magnetic keyed lock comprises a pushing member movably connected with the housing. The engagement member engages with the pushing member in the locked state of the magnetic keyed lock and releases the pushing member in the unlocked state of the magnetic keyed lock. The pushing member exerts a pushing force on the locking member in the unlocked state of the magnetic keyed lock. In particular, the door of the pump compartment springs open if the first and second protrusion disengage with the recess or with the opening of the locking member and of the pushing member, respectively. This increases user comfort.

In one embodiment, the at least one lock is a lock of a clamp of the infusion pump for fastening the infusion pump to a pole, and wherein the movable part is a clamp shaft of the clamp. The pole-lock mechanism makes removing the pump from pole more difficult or impossible, at least in a conventional hospital environment.

In one embodiment, the clamp is a screw clamp and the clamp shaft is a screw shaft arranged in a screw hole of the housing. A clamp jaw is provided e.g. as part of the housing, wherein the clamp jaw has a first arm and a second arm. The screw hole is provided in the first arm of the clamp jaw such that the screw shaft, when inserted in the screw hole, points with one end to the second arm of the clamp jaw. A clamping force can be exerted on a pole arranged in the space between the first and the second arm of the clamp jaw upon turning of the clamp shaft, i.e. screw shaft. A tip can be provided on one end of the screw shaft in order to limit damage to the pole upon clamping. Further, in some examples, a locking handle is provided on the opposite end of the screw shaft in order to alleviate rotation of the same.

In one embodiment, the locking member is a gear comprising at least one recess, wherein gear is arranged on the clamp shaft such that the rotational axis of the gear is aligned with the rotational axis of the clamp shaft. Then the engagement member, in particular the protrusion of the engagement member, can be released from its engagement with the at least one recess of the locking member, i.e. the gear, through placement of a magnetic key on or in the vicinity of the magnetic keyed lock, in particular through application of a defined magnetic field. Preferably, locking member and engagement member form a ratchet, wherein the engagement member functions as the prawl and the locking member as the gear of the ratchet. The engagement member is preferably spring loaded in order to provide the blocking force of the ratchet. The prawl can be removed from the gear, in particular from the at least one recess of the gear, through placement of a magnetic key on or in the vicinity of the magnetic keyed lock, in particular through application of a defined magnetic field. In doing so, the applied magnetic force has to overcome the blocking force of the ratchet, in particular the spring force exerted on the engagement member. For example, the engagement member comprises a first lever arm and a second lever arm, wherein a spring force is exerted on the first lever arm and wherein the second lever arm provides the protrusion of the engagement member. First and second lever arm are arranged at an angle. They are rotatably connected to the housing at their intersection point. In particular, the spring force exerted on the first lever arm holds the second lever arm in the at least one recess of the locking member, i.e. the gear, in the first position, i.e. in the locked state. Upon application of the defined magnetic field, the spring force is overcome and the engagement member rotated such that the second lever arm disengages from the at least one recess. The gear can move freely and the pump can be removed. E.g. the first lever is provided with at least one magnet and the magnetic key provides a repulsing force on that magnet.

In one embodiment, the housing comprises at least one recess for insertion of a magnetic key. The recess provides a receptacle for a key of the magnetic keyed lock. Alternatively, the housing comprises at least one through-hole for insertion of a magnetic key. A through-hole has the advantage that liquid and other debris can flow through. In another alternative embodiment, a guidance is provided on the housing in order to mark a placement area for the key. The placement area is the area on the surface of the housing on or in the vicinity of which a magnetic key has to be placed in order to unlock the magnetic keyed lock. Examples are a frame provided on the surface of the housing, e.g. protruding from the surface of the housing, or a graphical symbol such as a lock. With the help of the guidance, the user can easily identify the key placement area and thus, swiftly operate the lock. Also, a protruding frame brings additional robustness to the keyed solution, preventing the use as an example-of bigger magnets with the resulting increased airgap, such that only one specific key shape is able to fit into the frame.

The infusion pump is, in one embodiment, provided with both locks: a first lock securing the door of the pump compartment as well as a second lock securing the pump itself to a pole.

The objective is likewise achieved by means of a magnetic key for an infusion pump, wherein the magnetic key comprises a magnet which is configured to unlock the at least one magnetic keyed lock of the infusion pump, as well as a system of an infusion pump and such a magnetic key. In one embodiment, the magnetic key comprises a soft ferromagnetic element, for example an iron yoke. This soft ferromagnetic element increases the magnetic flux caused by the magnet. In one embodiment, the soft ferromagnetic element is arranged in the magnetic key such that it shields the magnet in order to reduce the magnetic flux escaping from the magnet in undesired directions.

Further, a method for unlocking a lock of an infusion pump according to an embodiment of the present invention with a magnetic key according to an embodiment of the present invention is disclosed, wherein the magnetic keyed lock is unlocked solely through placement of the magnetic key on the magnetic keyed lock or in the vicinity of the magnetic keyed lock. Placement of magnetic key on the magnetic keyed lock comprises in one example inserting the key in the at least one recess in the housing for insertion of a magnetic key or inserting the key in the at least one through-hole in the housing for insertion of a magnetic key or placing the key on a placement area of the housing. In contrast to prior art methods relying on mechanical locks, no turn of the key is required. This facilitates unlocking and hence, handling of the pump by authorized personal.

The idea underlying the invention shall subsequently be described in more detail with respect to the embodiment shown in the drawings. Herein:

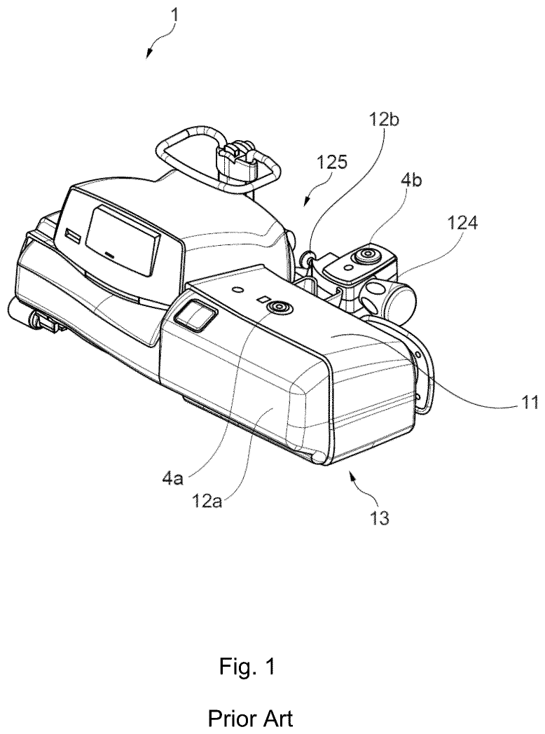

FIG. 1 shows an infusion pump according to the prior art;

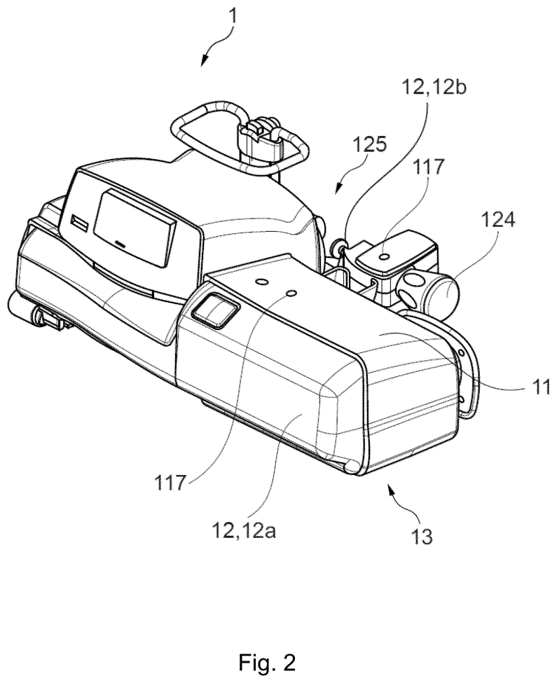

FIG. 2 shows an infusion pump according to an embodiment of the present invention;

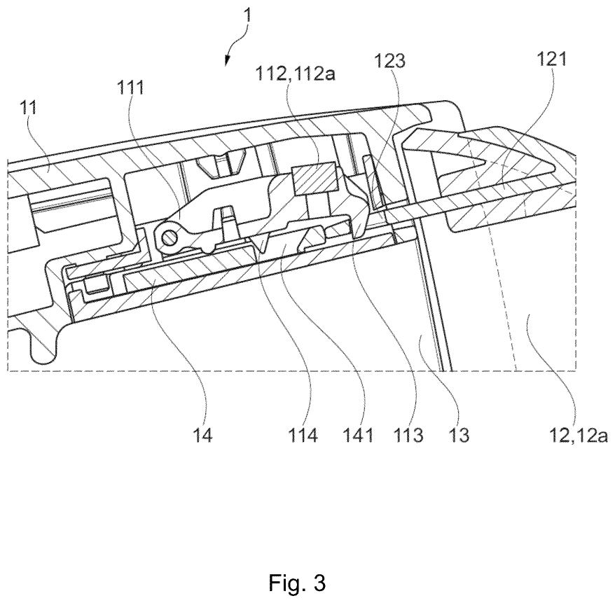

FIG. 3 shows a sectional side view of a part of an infusion pump according to an embodiment of the present invention, wherein a magnetic keyed lock of the pump is in a locked state;

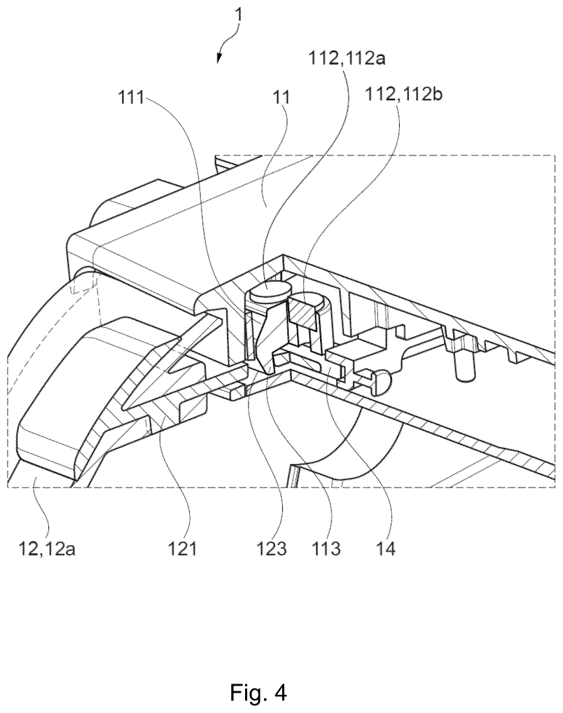

FIG. 4 shows a top-front view of the part of the infusion pump of FIG. 3 with the magnetic keyed lock in the locked state;

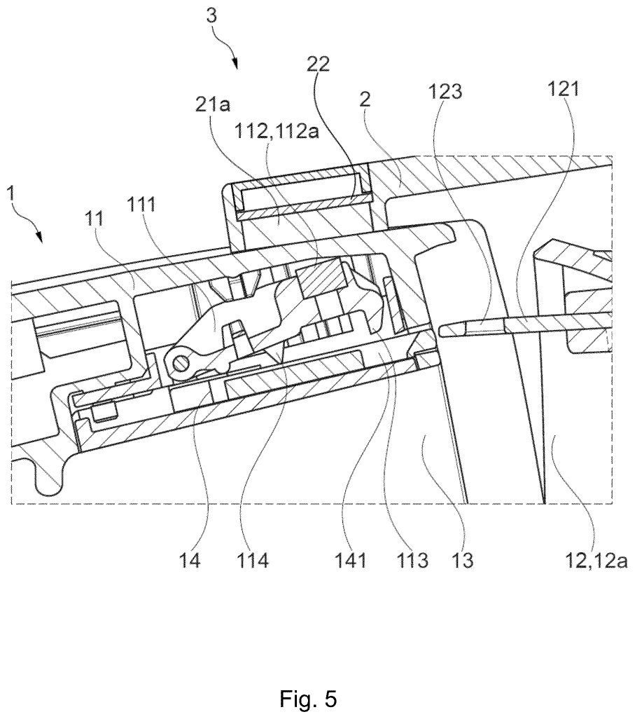

FIG. 5 shows a sectional side view of a system according to the present invention with the part of the infusion pump of FIG. 3, here with the magnetic keyed lock in an unlocked state, and with a magnetic key according to an embodiment of the present invention;

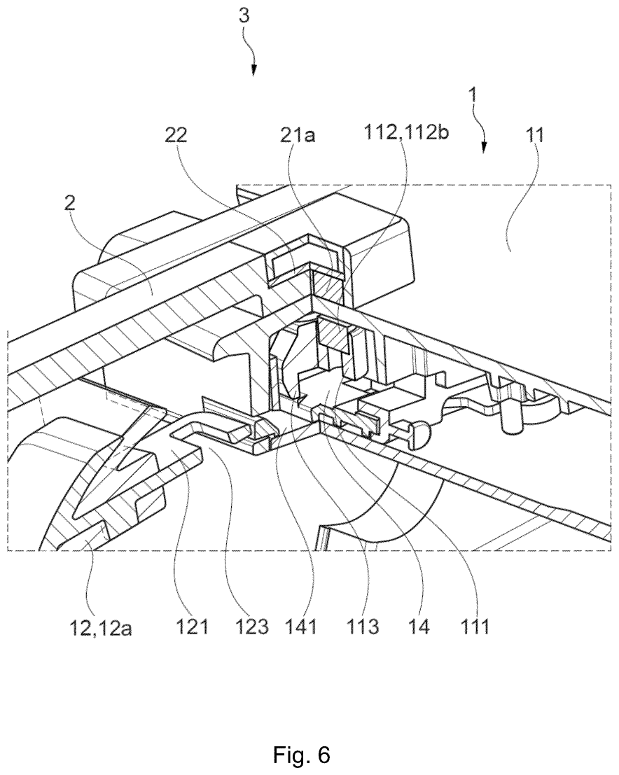

FIG. 6 shows a top-front view of the system of FIG. 5 with the magnetic keyed lock in the unlocked state;

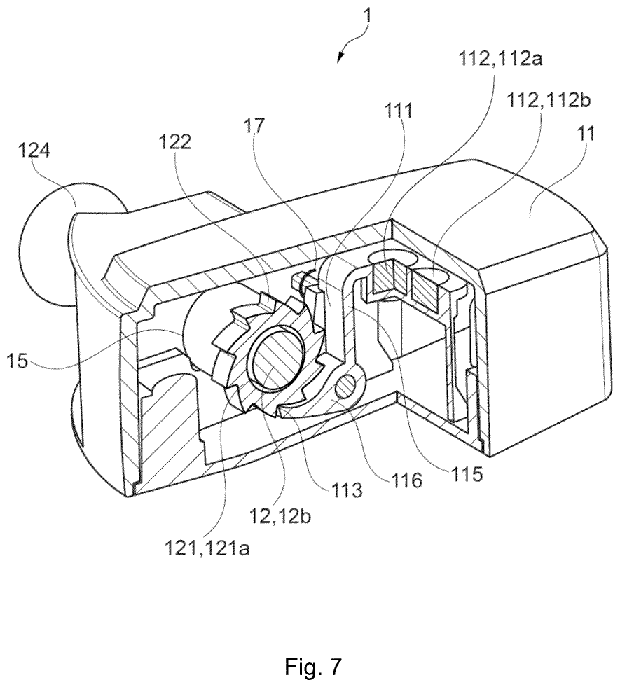

FIG. 7 shows a top-front view of a part of an infusion pump according to an embodiment of the present invention wherein the magnetic keyed lock of the pump is in a locked state;

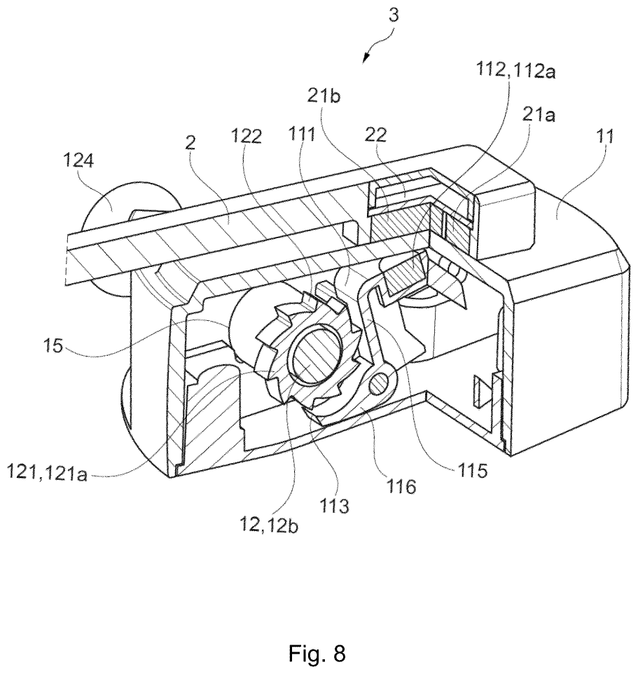

FIG. 8 shows a top-front view of a system according to the present invention with the part of the infusion pump of FIG. 7, here with the magnetic keyed lock in an unlocked state of the pump, and with a magnetic key according to an embodiment of the present invention;

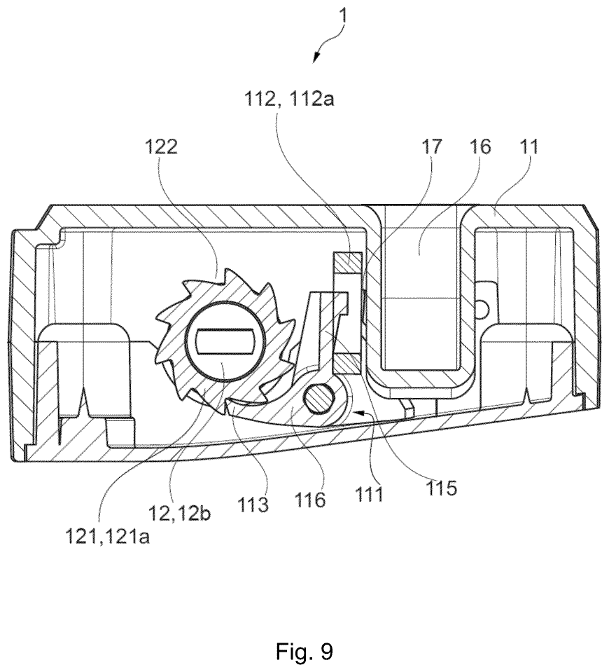

FIG. 9 shows a sectional view of a part of an infusion pump according to an embodiment of the present invention, wherein the magnetic keyed lock of the pump is in a locked state; and

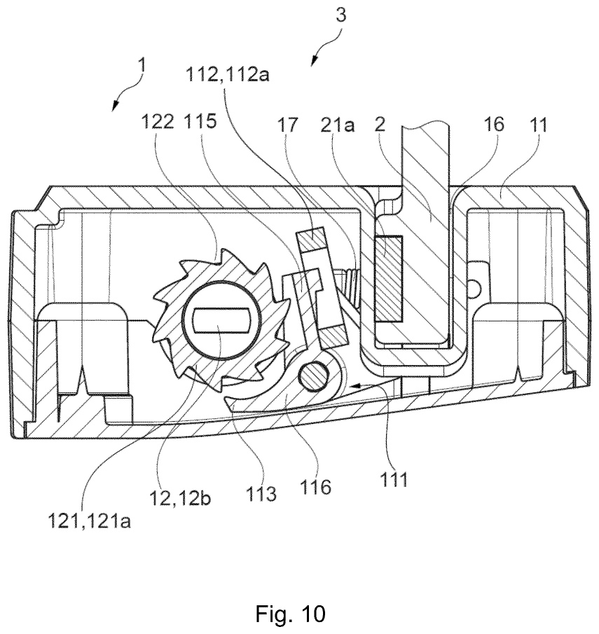

FIG. 10 shows a sectional view of a system according to the present invention with the part of the infusion pump of FIG. 9, here with the magnetic keyed lock in an unlocked state, and with a magnetic key according to an embodiment of the present invention.

FIG. 1 shows an infusion pump 1 according to the prior art. The infusion pump 1 comprises a housing 11. A compartment 13 is provided in the housing 11. In the pump compartment 13, a syringe can be arranged which provides medication, anaesthetics and/or analgesia for infusion. The compartment 13 has a door 12a. The pump 1 can be locked to a pole. To this end, a clamp jaw 125 is provided. Through rotation of a locking handle 124, a clamp shaft 12b is extended into the clamp jaw 125 such that the infusion pump 1 can be fixed to a pole. The infusion pump 1 comprises two mechanical locks 4a, 4b in order to prevent unauthorized access to any medication, anaesthetic and/or analgesic that is provided in the pump, in particular in a syringe arranged in the compartment 13.

First of all, a mechanical lock 4a is provided to prevent an unauthorized opening of the compartment door 12a. The mechanical lock 4a is provided on top of the compartment 13. Through insertion of a key and rotation of the key in the depicted direction, the mechanical lock 4a is unlocked. The door 12a can be opened and a syringe arranged in the compartment 13 accessed. A second mechanical lock 4b is provided in order to prevent a reverse rotation of the locking handle 124 and hence a loosening of the clamp jaw 125 from the pole. Hence, the infusion pump 1 can only the loosened from the pole by authorized personal. Through insertion of a key and rotation in the depicted direction, the mechanical lock 4b is unlocked, the handle 124 can be rotated such that the clamp shaft 12b is removed from the clamp jaw 125.

It is a disadvantage of the infusion pump 1 of the prior art, that the mechanicals locks 4a, 4b can be pried open through abuse of simple tools available in a hospital environment, such as e.g. a knife. Furthermore, liquid can enter the mechanical locks 4a, 4b which leads to corrosion. This reduces the durability of the mechanical locks 4a, 4b.

FIG. 2 shows an infusion pump 1 according to an embodiment of the present invention. The infusion pump 1 comprises a housing 11 providing a compartment 13. In the compartment 13, a syringe can be provided which contains, for example, medication, anaesthetics and/or analgesia. A movable part 12 of the housing 11, in the present embodiment a door 12a, closes the compartment 13. As in the prior art, the pump 1 is provided with a pole-lock mechanism, i.e. a mechanism to lock the pump 1 to a pole. To this end, a clamp jaw 125 is provided as part of the housing 11. Through rotation of a locking handle 124, a movable part 12 of the housing, in the present embodiment a clamp shaft 12b extends into the clamp jaw 125 such that the infusion pump 1 can be fixed to a pole.

In order to prevent unauthorized access to a syringe located in the pump compartment 13, a magnetic keyed lock is provided that locks the pump compartment 13. The magnetic keyed lock is unlocked through placement of the magnetic key 2 (see e.g. FIG. 5) on or in the vicinity of the magnetic keyed lock. A guidance 117 marks a placement area for placement of the magnetic key 2 such as to guide the user.

In order to prevent unauthorized removal of the infusion pump 1 from a pole, a second magnetic keyed lock is provided that prevents a reverse rotation of the clamp shaft 12b.

Through approach of a magnetic key 2, e.g. the same key used for unlocking the magnetic keyed lock of the pump compartment 13, the clamp shaft 12b is released and the clamp jaw 125 can be loosened. The pump 1 can be removed from the pole. Here, likewise a guidance 117 is used in order to mark the placement area for a magnetic key 2.

In contrast to conventional, mechanical locks, the magnetic keyed lock cannot be pried open through the abuse of simple tools available in a hospital environment, such as knifes. Moreover, no opening in the housing 11 for a keyhole needs to be provided, such that the lock is liquid-proof and hygienic. Corrosion is thus prevented and the durability of the pump 1 increased.

FIG. 3 shows a part of an infusion pump 1 according to an embodiment of the present invention in a side sectional view of the pump 1. In particular, FIG. 3 illustrates an infusion pump 1 with a lock for a pump compartment 13 of the infusion pump 1. The pump compartment 13 is part of the housing 11 of the pump 1. The pump compartment 13 can be accessed through a movable part 12 of the housing 11, in the present embodiment a door 12a, arranged in the front of the pump 1, in particular its housing 11. The door 12a can be opened and hence, a syringe provided in the pump compartment 13 be accessed. In order to prevent unauthorized access to the pump compartment 13, its door 12a is secured by a magnetic keyed lock. The magnetic keyed lock comprises an engagement member 111 which is connected to the housing 11. The magnetic keyed lock further comprises a locking member 121 which is connected with the door 12a. The engagement member 111 engages with the locking member 121 in a first position which corresponds to a locked state of the magnetic keyed lock. In one embodiment, the engagement member 111 is kept in its first position through an external force, e.g. a spring force exerted by a suitable arranged spring (not shown) provided inside the housing 11 as part of the magnetic keyed lock. In particular, the engagement member 111 comprises a first protrusion 113 which engages with an opening 123 of the locking member 121. Hence, through the interlocking of engagement member 111 and locking member 121, the door 12a is secured to the housing 11 and cannot be opened. It is locked. The engagement member 111 further comprises at least one ferromagnetic element 112. The ferromagnetic element 112 may be a permanent magnet or simply an element comprising a soft ferromagnetic material. Important is that it reacts to an external magnetic field. In the presently depicted embodiment, two hard ferromagnetic elements 112 are provided in the form of two permanent magnets 112a, 112b. Through the arrangement of multiple permanent magnets as hard ferromagnetic elements, a specific magnetic pattern can be generated. For instance, an upward magnet polarization on one element is combined with a downward polarization on the other one. If a magnetic key 2 is placed on or in the vicinity of the magnetic keyed lock, a defined magnetic field is applied, and the engagement member 111 moves from the displayed first position to a second position, corresponding to the unlocked state of the magnetic keyed lock which will be discussed with regard to FIG. 5. As long as the magnetic key 2 is absent, i.e. defined magnetic field is absent, the engagement member 111 rests in the first position. Thus, it secures the door 12a to the housing 11 as long as no magnetic key 2 is placed on or in the vicinity of the magnetic keyed lock. In the illustrated embodiment, the magnetic keyed lock further comprises a pushing member 14 which is movably connected with the housing 11. In the first position corresponding to the locked state of the magnetic keyed lock, the engagement member 111 also engages with the pushing member 14. For example, the engagement member 111 comprises a second protrusion 114 which engages with an opening 141 of the pushing member 14.

FIG. 4 shows a part of the infusion pump 1 according to the same embodiment as FIG. 3, but from a top-front view in order to facilitate understanding. In this view, the arrangement of the two magnets 112a, 112b can be seen.

FIG. 5 illustrates a system 3 of key 2 and infusion pump 1. Shown is the part of the infusion pump 1 according to the same embodiment of the infusion pump 1 as FIG. 3 in a side sectional view, but now in an unlocked state of the magnetic keyed lock of the pump 1. The engagement member 111 releases the locking member in the unlocked state of the magnetic keyed lock displayed here. As the engagement member 111 is movably, in particular rotatably, connected to the housing 11, it switches from the first position displayed in FIGS. 3 and 4 to a second position displayed here, corresponding to the unlocked state of the magnetic keyed lock. In particular, the engagement member 111 rotates into the second position. The switch from the first into the second position is achieved through the application of a defined magnetic field. Hence, the magnetic keyed lock is opened through the application of the defined magnetic field. The defined magnetic field is the specific field necessary in order to achieve the switch of the engagement member from the first position to the second position and hence, the switch from the locked to the unlocked state of the magnetic keyed lock. This requires a certain strength of the defined magnetic field and/or a certain magnetic pattern of the defined magnetic field. The defined magnetic field is applied through placement of a suitable magnetic key 2 on or in the vicinity of the magnetic keyed lock, wherein the magnetic key 2 comprises at least one magnet 21a. The magnet 21a generates the defined magnetic field at the location of the at least one ferromagnetic element 112, in the present embodiment two permanent magnets 112a, 112b. The engagement member 111 is hence subjected to a magnetic force that overcomes the forces that held the engagement member 111 in its first position. These may be gravitational forces and, in one embodiment, a spring force provided by a suitably arranged spring (not shown). Under the influence of the defined magnetic field, i.e. through the interaction of the magnets 112a, 112b with the magnet 21a, the engagement member 111 rotates. In the illustrated embodiment, the engagement member 111 rotates from the first into the second position under the pulling force of the defined magnetic field. The attraction between the magnet 21a of the magnetic key 2 and the magnets 112a, 112b moves, i.e. rotates, the engagement member 111 from the first into the second position. The first protrusion 113 is removed from the opening 123 of the locking member 121. The locking member 121 no longer engages with the engagement member 111. The door 12a can be opened. As the pushing member 14 is being released through the disengagement of the first protrusion 113, the pushing member 14 further pushes on the second protrusion 114, adding to the rotation of the engagement member 111. The pushing member 14 exerts a force onto the locking member 121 and hence, pushes the door 12a open. The pump compartment 13 can be accessed. With the pump 1 and magnetic key 2, a system 3 is provided that restricts access to the pump compartment 13 and, hence, increases patient safety. In the depicted embodiment, additionally, a soft ferromagnetic element 22, e.g. an iron yoke, is provided next to the magnet 21a of the key 2 in order to shield the magnet 21a. In particular, the soft ferromagnetic element 22 is provided on the side of the magnet 21a which faces away from the magnets 112a, 112b when placing the magnetic key 2 on or in the vicinity of the magnetic keyed lock.

FIG. 6 shows the system of FIG. 5 with the magnetic keyed lock in its unlocked state, but from a top-front view in order to facilitate understanding. As can be seen, in the second position, the engagement member 111 releases the locking member 121. In fact, the locking member 121 is pushed by the pushing member 14, hence effecting an opening of the door 12a. In this embodiment, the locking member 111 is part of a slider that is provided on the door 12a. Through movement of the slider, the door 12a can be opened if the magnetic keyed lock is in its unlocked state, i.e. the engagement member 111 in its second position. In the displayed embodiment, the magnetic key 2 comprises a handle and a head, wherein the magnet 21a is arranged in the head of the magnetic key 2. In the depicted embodiment, additionally, a soft ferromagnetic element 22, e.g. an iron yoke, is provided next to the magnet 21a in order to shield the magnet 21a. In particular, the soft ferromagnetic element 22 is provided on the side of the magnet 21a which faces away from the two magnets 112a, 112b when placing the key 2 on the housing 11 in order to open the lock. Placing the magnetic key 2 on or in the vicinity of the magnetic keyed lock comprises placing the magnetic key 2, in particular the magnet 21a of the key, on or in the vicinity of the part of the housing 11 where the magnetic keyed lock, in particular the magnets 112a, 112b, are located. No opening in the housing 11 needs to be provided. No turning of the key 2 is necessary. This facilitates handling and reduces space requirements. In particular, the horizontal approach of the key 2 to the top of the housing 11 facilitates handling and improves the user experience in cases where the space above the pump 1 is restricted, e.g. when multiple pumps 1 are arranged on top of each other on a single pole or on a rack, e.g. of an infusion pump station or stacked on top of each other otherwise.

FIG. 7 shows a part of an infusion pump 1 according to an embodiment of the present invention in a top-front view. In particular, FIG. 7 illustrates a part of an infusion pump 1 with a lock for a clamp of the pump 1 for fastening the pump 1 to a pole. For example, a clamp jaw 125 (see e.g. FIG. 2) is provided as part of the housing 11. The clamp jaw 125 has a first arm and a second arm. A screw hole 15 is provided in the first arm of the clamp jaw 125 such that a movable part 12 of the housing 11, in the present embodiment a clamp shaft 12b, when inserted in the screw hole 15, points with one end to the second arm of the clamp jaw 125. The clamp shaft 12b is provided as a screw shaft, i.e. with an external thread. A clamping force can be exerted on a pole arranged in the space between the first and the second arm of the clamp jaw 125 upon turning of the clamp shaft 12b. A tip can be provided on one end of the clamp shaft 12b in order to limit damage to the pole upon clamping. Further, in some examples, a locking handle 124 (only its tip shown in the depicted view) is provided on the opposite end of the clamp shaft 12b in order to alleviate rotation of the same. The magnetic keyed lock comprises a locking member 121, in the present embodiment a gear 121a. The gear 121a comprises at least one recess 122. The engagement member 111, in particular its first protrusion 113, engages with the at least one recess 122 of the gear 121a in a first position. This first position corresponds to a locked state of the magnetic keyed lock. A release of the clamp jaw 215, and hence a removal of the pump 1 from the pole, is not possible in this first position of the engagement member 111. In this particular embodiment, the engagement member 111 comprises a first lever arm 115 and a second lever arm 116, wherein the first protrusion 113 is provided as part of the second lever arm 116. In particular, engagement member 111 and gear 121a form a ratchet. The teeth of the gear 121a and the second lever arm 116, in particular the part providing the first protrusion 113, are formed such that the clamp shaft 12b can be turned in one direction, but is blocked in the opposite direction. Hence, the clamp jaw 125 can be tightened, but not loosened while the engagement member 111 is in its first position, i.e. the first protrusion 113 engages with one of the recesses 122 of the gear 121a. The engagement member 111 is movably connected with the housing 11. In particular, the engagement member 111 is rotatably connected with the housing 11 such that the engagement member 111 can move, in particular rotate, from its first position to a second position. This second position corresponds to an unlocked state of the magnetic keyed lock which will be further discussed with regard to FIG. 8. In the displayed embodiment, first and second lever arm 115, 116 intersect and the engagement member 111 is connected to the housing 11 at their intersection point. For example, the engagement member 111 is v-shaped with the two legs of the “v” forming the first and second lever arms 115, 116, respectively. E.g. a bolt is provided that connects the engagement member 111 with the housing 11 at the intersection point. Then, a rotation of the engagement member 111 is possible, wherein the axis of rotation is perpendicular to the plane spanned by the first and second lever arms 115, 116. The engagement member 111 comprises at least one ferromagnetic element 112, in the present example two magnets 112a, 112b with opposed magnetic polarities. The engagement member 111 may additionally be held in its position through a spring force applied by a suitably arranged spring 17. Through placement of a suitable magnetic key 2 on or in the vicinity of the magnetic keyed lock, i.e. through the application of a defined magnetic field, the engagement member 111 switches from the displayed first position to a second position corresponding to the unlocked state of the magnetic keyed lock. The spring force exerted on the engagement member 111, in particular its first lever arm 115, has to be overcome by the applied magnetic force in order to effect this switch.

FIG. 8 illustrates a system 3 of magnetic key 2 and infusion pump 1. It shows the part of the pump of FIG. 7 with the magnetic keyed lock in its unlocked state. Through the approach of a suitable magnetic key 2, the magnetic keyed lock is opened, i.e. switched from its locked into its unlocked state. The magnetic key 2 comprises at least one magnet 21a. In the present example, two magnets 21a, 21b are provided. These magnets 21a, 21b are for example provided in a head of the magnetic key 2, which is located at the end of a key handle. This allows comfortable handling while unlocking the magnetic keyed lock. In the depicted embodiment, additionally, a soft ferromagnetic element 22, e.g. an iron yoke, is provided next to the magnets 21a, 21b in order to shield the magnets 21a, 21b. In particular, the soft ferromagnetic element 22 is provided on the side of the magnets 21a, 21b which faces away from the magnets 112a, 112b when placing the magnetic key 2 on the housing 11 in order to unlock the lock. In the present embodiment, the placement area for placing the magnetic key 2 is at the top of the housing 11, enabling easy access, in particular when space above the pump 1 is limited because multiple pumps 1 are arranged on top of each other on a pole or in a rack, e.g. of an infusion pump station, or stacked on top of each other otherwise. The magnets 21a, 21b provide the defined magnetic field at the location of the magnets 112a, 112b. Hence, a force is exerted on the magnets 112a, 112b, and thus also on the engagement member 111, moving it from its first into its second position. In the present example, the force is an attractive force, effecting a rotation of the engagement member 111. In one embodiment, the force exerted by the magnets 21a, 21b has to be large enough to overcome the spring force exerted upon the engagement member 111, in particular its first lever arm 115, by a spring 17. The first lever arm 115 is pulled towards the magnetic key 2 and the second lever arm 116 is, as a result, pushed away from the gear 121a. The first protrusion 113 is removed from the corresponding recess 122 of the gear 121a. The gear 121a can rotate freely. Consequently, the clamp shaft 12b can be rotated in the opposite direction, thus loosening the clamp jaw 125 from the pole. The infusion pump 1 hence can be removed.

FIG. 9 shows a part of an infusion pump 1 according to an alternative embodiment of the present invention in a sectional view. In particular, FIG. 9 illustrates an infusion pump 1 with a lock for a clamp of the pump 1 for fastening the pump 1 to a pole. The infusion pump of this FIG. 9 is largely the same as the infusion pump and lock explained with regard to FIGS. 7 and 8. In the present embodiment, the at least one ferromagnetic element 112, in particular a magnet 112a, is provided at the rear of first lever arm 115. That means, the ferromagnetic element 112, in the present embodiment a magnet 112a, is provided on the side of the first lever arm 115 that faces away from the gear 121a. The first lever arm 115 is spring loaded with a traction spring 17. The at least one ferromagnetic element 112, i.e. the magnet 112a, is recalled by the traction spring 17, hence keeping the engagement member 111 in its first position corresponding to the locked position of the magnetic keyed lock. A recess 16 is provided in the housing 11 for the insertion of a magnetic key 2. The recess 16 is provided such that an inserted magnetic key 2 comes into close proximity to the magnet 112a.

FIG. 10 illustrates a system 3 of a magnetic key 2 and an infusion pump 1 according to an embodiment of the present invention. It shows a part of the infusion pump 1 of FIG. 9 with the magnetic keyed lock in the unlocked state. Upon insertion of the suitable magnetic key 2 with magnet 21a in the recess 16, a defined magnetic field is applied to the magnet 112a. Hence, the engagement member 111 rotates under the ensuing magnetic force into its second position. The gear 121a can move freely. The magnetic keyed lock is unlocked. The magnet 21a exerts a repulsive force on the magnet 112a. The first lever 115 is pushed towards the gear 121a, the second lever 116 consequently removed from the gear 121a. No turn of the key 2 is necessary. The unlocking is achieved through mere insertion of the key 2 in the corresponding recess 16. The recess 16 is formed in the housing 11. Hence, no keyhole has to be provided in the housing 11. The pump 1 is kept liquid-prove. No corrosion of the lock can occur. In an alternative embodiment (not shown), the recess 16 is replaced by a through-hole. This still provides a receptacle for the magnetic key 2, but allows liquid to flow through.

### LIST OF REFERENCE NUMERALS

1 infusion pump
11 housing
111 engagement member
112 ferromagnetic element
112a, 112b magnet
113 first protrusion
114 second protrusion
115 first lever arm
116 second lever arm
117 guidance
12 moveable part of pump
12a door
12b clamp shaft
121 locking member
121a gear
122 recess of locking member
123 opening of locking member
124 locking handle
125 clamp jaw
13 pump compartment
14 pushing member
141 recess or opening of pushing member
15 screw hole
16 recess for insertion of a magnetic key
17 spring
2 magnetic key
21a, 21b magnet
22 soft ferromagnetic element of the magnetic key
3 system
4a, 4b mechanical lock

1. An infusion pump for patient-controlled analgesic treatment, the infusion pump comprising at least one lock preventing unauthorized handling of the infusion pump, the at least one lock being a magnetic keyed lock, wherein the magnetic keyed lock has a locked state and an unlocked state and wherein the magnetic keyed lock is configured to switch from the locked state to the unlocked state through placement of a magnetic key on the magnetic keyed lock or in the vicinity of the magnetic keyed lock.

2. The infusion pump according to claim 1, further comprising a housing and a movable part, and wherein the magnetic keyed lock comprises an engagement member connected with the housing of the infusion pump and wherein the magnetic keyed lock comprises a locking member connected with the movable part of the infusion pump, wherein the engagement member engages with the locking member in the locked state of the magnetic keyed lock and releases the locking member in the unlocked state of the magnetic keyed lock.

3. The infusion pump according to claim 2, wherein the engagement member comprises at least one ferromagnetic element, wherein the at least one ferromagnetic element comprises at least one of a hard ferromagnetic material and a soft ferromagnetic material.

4. The infusion pump according to claim 2, wherein the engagement member is movably connected to the housing and configured so that the engagement member switches between a first position corresponding to the locked state of the magnetic keyed lock and a second position corresponding to the unlocked state of the magnetic keyed lock through placement of a magnetic key on the magnetic keyed lock or in the vicinity of the magnetic keyed lock.

5. The infusion pump according to claim 4, wherein the engagement member is rotatably connected to the housing and configured so that the engagement member is rotatable between the first position and the second position.

6. The infusion pump according to claim 4, wherein the engagement member rests in the first position, when no magnetic key is placed on the magnetic keyed lock or placed in the vicinity of the magnetic keyed lock, and wherein the engagement member switches into the second position, when a magnetic key is placed on the magnetic keyed lock or in the vicinity of the magnetic keyed lock.

7. The infusion pump according to claim 4, wherein the engagement member comprises a first protrusion and wherein the locking member comprises at least one recess or at least one opening, wherein the first protrusion engages with the at least one recess or with the at least one opening in the locked state of the magnetic keyed lock and wherein the first protrusion is removed from the at least one recess or from the or at least one opening in the unlocked state of the magnetic keyed lock.

8. The infusion pump according to claim 2, wherein the at least one lock is a lock of a pump compartment of the infusion pump wherein the pump compartment is part of the housing of the infusion pump and the movable part is a door of the pump compartment.

9. The infusion pump according to claim 8, wherein the magnetic keyed lock comprises a pushing member movably connected with the housing and wherein the engagement member engages with the pushing member in the locked state of the magnetic keyed lock and wherein the engagement member releases the pushing member in the unlocked state of the magnetic keyed lock and wherein the pushing member exerts a pushing force on the locking member in the unlocked state of the magnetic keyed lock.

10. The infusion pump according to claim 2, wherein the at least one lock is a lock of a clamp of the infusion pump for fastening the infusion pump to a pole, and wherein the movable part is a clamp shaft of the clamp.

11. The infusion pump according to claim 10, wherein the clamp is a screw clamp and wherein the clamp shaft is a screw shaft arranged in a screw hole of the housing.

12. The infusion pump according to claim 11, wherein the locking member is a gear comprising at least one recess, wherein the gear is arranged on the clamp shaft with a rotational axis of the gear aligned with a rotational axis of the clamp shaft, the locking member and the engagement member forming a ratchet.

13. The infusion pump according to claim 1, further comprising a housing, the housing comprising at least one recess for insertion of a magnetic key or a through-hole for insertion of a magnetic key or the housing comprising a guidance that marks a placement area for the magnetic key.

14. A magnetic key for an infusion pump according to claim 1, wherein the magnetic key comprises at least one magnet which is configured to unlock the magnetic keyed lock of the infusion pump.

15. A system comprising an infusion pump according to claim 1 and a magnetic key comprising at least one magnet which is configured to unlock the magnetic keyed lock of the infusion pump.

16. A method for unlocking a lock of an infusion pump according to claim 1 with a magnetic key comprising at least one magnet which is configured to unlock the magnetic keyed lock of the infusion pump, wherein the magnetic keyed lock is unlocked solely through placement of the magnetic key on the magnetic keyed lock or in the vicinity of the magnetic keyed lock.
